(19) United States
(12) Patent Application Publication
(10) Pub. No.: US 2026/0289877 A1
(43) Pub. Date: Sep. 24, 2026

(54) IMAGE PROCESSING METHOD, IMAGE PROCESSING APPARATUS, AND X-RAY CT APPARATUS

(71) Applicant: CANON MEDICAL SYSTEMS CORPORATION, Tochigi (JP)

(72) Inventors: Zhao LONGFEI, Beijing (CN); Xu QIQI, Beijing (CN); Han BING, Beijing (CN)

(73) Assignee: CANON MEDICAL SYSTEMS CORPORATION, Tochigi (JP)

(21) Appl. No.: 19/570,440

(22) Filed: Mar. 18, 2026

(30) Foreign Application Priority Data
Mar. 19, 2025 (CN) 202510325784.3

(51) Int. Cl. G06T 12/20; G06T 7/00; G06T 7/10; G06T 15/08; G06V 10/25; G16H 30/40

(52) U.S. Cl. CPC G06T 12/20; G06T 7/0012; G06T 7/10; G06T 15/08; G06V 10/25; G16H 30/40; G06T 2207/30101

(57) ABSTRACT

An image processing method according to an embodiment includes: a point data generation step of generating, on the basis of three-dimensional volume data including a region of interest, three-dimensional point data representing the region of interest; and an inference step of inferring the region of interest in the three-dimensional volume data, by inputting the three-dimensional volume data and the three-dimensional point data as prompts into an inference model.

### CROSS-REFERENCE TO RELATED APPLICATIONS

This application is based upon and claims the benefit of priority from Chinese Patent Application No. 202510325784.3, filed on Mar. 19, 2025; the entire contents of all of which are incorporated herein by reference.

### FIELD

Embodiments disclosed in the present specification and the drawings relate to image processing methods, image processing apparatuses, and X-ray CT apparatuses.

### BACKGROUND

Segmentation techniques for medical images are important components of image processing methods for medical images. Their main objective is to accurately segment and extract a target structure having a specific enhanced signal from a complex medical image and to use the extracted target structure for subsequent tasks, such as image analysis or image measurement of the medical image. An enhanced signal of a target structure in a medical image may appear as a high-intensity signal (for example, a blood vessel enhanced with a contrast agent) or a low-intensity signal (for example, air in the trachea) relative to surrounding structures. To provide clinical applications, such as evidence, surgical navigation, and therapeutic effect evaluation, for clinical diagnosis, segmentation techniques in image processing methods for medical images are required to accurately segment and extract target structures. However, conventional image processing methods are typically capable of processing only enhanced signals having simple morphology, and are not capable of accurately segmenting and extracting enhanced signals having fine geometric structures or enhanced signals having complex morphology.

Specifically, image processing methods used for conventional segmentation tasks for medical images typically employ conventional deep learning methods, such as convolutional neural networks (CNNs) and Transformer architectures. However, there has been a problem in that in image processing methods based on conventional deep learning methods, when segmentation tasks for medical images are executed, generalization capability of models based on the conventional deep learning methods is insufficient, such methods typically enable only application of a specific model to a data domain distribution of a specific anatomical structure (for example, a certain tubular segmentation task), and rapidly applying the specific model to a new task or a new data domain distribution is difficult.

### BRIEF DESCRIPTION OF THE DRAWINGS

FIG. 1 is a block diagram illustrating an example of a configuration of an image processing system according to an embodiment;

FIG. 2 is a block diagram illustrating an example of a configuration of an image processing apparatus according to a first embodiment;

FIG. 3 is a flowchart illustrating a flow of an image processing method according to the first embodiment;

FIG. 4A is a diagram illustrating differences in evaluation metrics between the first embodiment and a comparative example;

FIG. 4B is a diagram illustrating differences in segmentation results between the first embodiment and the comparative example;

FIG. 5 is a diagram illustrating differences in segmentation results between the first embodiment and the comparative example;

FIG. 6 is a diagram illustrating differences in evaluation metrics between the first embodiment and the comparative example;

FIG. 7 is a block diagram illustrating an example of a configuration of an image processing apparatus according to a second embodiment;

FIG. 8 is a flowchart illustrating a flow of an image processing method according to the second embodiment;

FIG. 9 is a block diagram illustrating an example of a configuration of an image processing apparatus according to a third embodiment;

FIG. 10 is a flowchart illustrating a flow of an image processing method according to the third embodiment; and

FIG. 11 is a diagram illustrating an example of a configuration of an X-ray CT apparatus according to a fourth embodiment.

### DETAILED DESCRIPTION

One of problems to be solved by embodiments disclosed in this specification and the drawings is to improve inference efficiency. However, the problems to be solved by the embodiments disclosed in the specification and drawings are not limited to the above mentioned problem. Problems corresponding to respective effects of configurations described later with respect to the embodiments may also be regarded as other problems.

An image processing method according to an embodiment includes: a point data generation step of generating, on the basis of three-dimensional volume data including a region of interest, three-dimensional point data representing the region of interest; and an inference step of inferring the region of interest in the three-dimensional volume data, by inputting the three-dimensional volume data and the three-dimensional point data as prompts into an inference model. The region of interest has an enhanced signal.

As a result, application to segmentation tasks for complex enhanced signals is enabled, training time and cost are able to be reduced, and generalization capability of the inference model is able to be improved.

Furthermore, the three-dimensional point data are used as the prompts for the inference model. Therefore, a problem that annotation work for complex enhanced signals requires a long time, high cost, and a large amount of data is able to be avoided. At the same time, automation of a training process and an inference process of the inference model is achieved, manual labor time and cost are able to be reduced, and inference efficiency is able to be improved.

Furthermore, even if a segmentation task for complex enhanced signals of, for example, blood vessels and the trachea, is executed, high segmentation capability and accuracy are achieved.

In the image processing method according to the embodiment further includes a volume data processing step including: a projection image generation step of generating, on the basis of the region of interest in the three-dimensional volume data, a projection image that is two-dimensional and buffer data corresponding to the projection image; and a segmentation step of segmenting the region of interest in the projection image. In the point data generation step, the three-dimensional point data are generated on the basis of the three-dimensional volume data that have been processed in the volume data processing step.

In the image processing method according to the embodiment, the buffer data include an intensity signal corresponding to the projection image in the three-dimensional volume data.

In the image processing method according to the embodiment, in the point data generation step, on the basis of the buffer data, a segmentation result for the region of interest in the projection image is mapped to a three-dimensional space of the three-dimensional volume data to generate the three-dimensional point data.

The image processing method according to the embodiment further includes a preprocessing step of preprocessing the three-dimensional volume data and using the three-dimensional volume data that have been preprocessed and the three-dimensional point data as the prompts.

In the image processing method according to the embodiment, in the preprocessing step, the region of interest is segmented from the three-dimensional volume data, and an interference signal is removed therefrom.

In the image processing method according to the embodiment, in the preprocessing step, the region of interest is segmented using prior knowledge, a segmentation algorithm, or an AI algorithm.

In the image processing method according to the embodiment, in the preprocessing step, the interference signal is removed by using a threshold method or an AI algorithm.

In the image processing method according to the embodiment, in the projection image generation step, the projection image is generated by using at least one of: generating the projection image in each of a plurality of directions; generating the projection image by setting a slice interval; and generating the projection image in the region of interest.

In the image processing method according to the embodiment, in the segmentation step, the region of interest is segmented by using 2D Med SAM.

The image processing method according to the embodiment further includes a region-of-interest designation step of designating the region of interest in the three-dimensional volume data on the basis of a user's operation.

Segmentation tasks are thereby able to be executed even more accurately. This is because, on the basis of a user's operation, regions, such as microvessels, which may be undersegmented in the three-dimensional volume data, are designated as regions of interest, and the inference model is able to be caused to process these regions of interest intensively. Regions that may be undersegmented, such as microvessels, are thereby able to be segmented well.

In the image processing method according to the embodiment, the inference model is a large-scale segmentation model based on prompt engineering.

In the image processing method according to the embodiment, the enhanced signal of the region of interest is a signal having a structural intensity lower or higher than that of surrounding signals.

In the image processing method according to the embodiment, the projection image is a maximum intensity projection or a minimum intensity projection.

In the image processing method according to the embodiment, in the point data generation step, a center line is extracted from the three-dimensional volume data, and the three-dimensional point data are generated on the basis of the center line.

As a result, automation of a training process and an inference process of the inference model is able to be improved further, manual labor time and cost are able to be reduced, and inference efficiency is able to be improved.

In the image processing method according to the embodiment, in the point data generation step, the three-dimensional point data are arranged in the region of interest.

In the image processing method according to the embodiment, in the point data generation step, the region of interest includes a blood vessel, and the three-dimensional point data are arranged along the blood vessel.

In the image processing method according to the embodiment, in the point data generation step, the region of interest includes a blood vessel, a branch point of the blood vessel is treated as a node, and the three-dimensional point data are arranged at an end of the blood vessel.

In the image processing method according to the embodiment, the buffer data include information of the three-dimensional volume data, the information being along a first direction orthogonal to the projection image, and in the point data generation step, the three-dimensional point data are generated by mapping the segmentation result to the three-dimensional space by using the information of the three-dimensional volume data included in the buffer data, the information being along the first direction.

An image processing apparatus according to an embodiment includes: a point data generation unit that generates, on the basis of three-dimensional volume data including a region of interest, three-dimensional point data associated with the region of interest; and an inference unit that infers the region of interest in the three-dimensional volume data, by inputting the three-dimensional volume data and the three-dimensional point data as prompts into an inference model.

A storage medium according to an embodiment is a non-transitory computer-readable storage medium that stores a program for causing a computer to execute: a point data generation process of generating, on the basis of three-dimensional volume data including a region of interest, three-dimensional point data representing the region of interest; and an inference process of inferring the region of interest in the three-dimensional volume data, by inputting the three-dimensional volume data and the three-dimensional point data as prompts into an inference model.

An X-ray CT apparatus according to an embodiment includes: a point data generation unit that generates, on the basis of three-dimensional volume data including a region of interest, three-dimensional point data associated with the region of interest; and an inference unit that infers the region of interest in the three-dimensional volume data, by inputting the three-dimensional volume data and the three-dimensional point data as prompts into an inference model.

Embodiments of an image processing method, an image processing apparatus, a storage medium, and an X-ray computed tomography (CT) apparatus according to the present disclosure will be described hereinafter by reference to the drawings.

Cases where image processing methods, image processing apparatuses, and programs, according to the embodiments are applied to blood vessel segmentation tasks for the liver and lungs will be described hereinafter as examples. This is because blood vessels in the liver and lungs generally have a complex and fine tree structure and there is a problem that annotation cost is high, difficulty of presentation is large, and segmenting microvessels is difficult. However, the embodiments are not limited to such cases, and it goes without saying that the embodiments are also applicable to segmentation tasks for other tissues, organs, and structures.

### First Embodiment

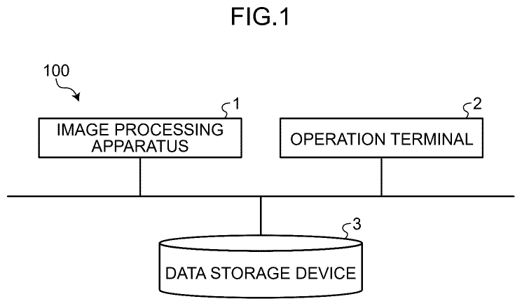

An example of a configuration of an image processing system 100 will be described by reference to FIG. 1. FIG. 1 is a block diagram illustrating the example of the configuration of the image processing system 100 according to a first embodiment.

The image processing system 100 includes an image processing apparatus 1, an operation terminal 2, and a data storage device 3. These devices are connected so as to be capable of communicating with one another via a common signal transmission path, for example, a bus or a network.

The image processing apparatus 1 is an apparatus that executes segmentation tasks for medical images. For example, the image processing apparatus 1 functions as a server of the image processing system 100 and may be a workstation capable of executing high-speed information processing.

The operation terminal 2 is a terminal operated by a user. The operation terminal 2 functions as a client of the image processing system 100. The operation terminal 2 may be, for example, a computer, a notebook computer, a smartphone, a tablet or wearable terminal, or a mobile or handheld terminal.

Furthermore, the operation terminal 2 may have an input interface that receives various operation from a user. The input interface converts various operation received from an operator into electrical signals and transmits the electrical signals to processing circuitry of the operation terminal 2. The input interface of the operation terminal 2 may be a mouse, a keyboard, a button, a panel switch, a slider switch, a trackball, an operation panel, or a touch panel.

Furthermore, the operation terminal 2 may have a display to display various data and information. The display may be a liquid crystal display, a plasma display, an organic electroluminescence display (OLED), or an LED display. The display may be a touch panel display also having a function of an input interface.

For example, a user performs operation to designate a region of interest described later, by operating the operation terminal 2.

The data storage device 3 is a device to store medical image data. The data storage device 3 stores, for example, medical image data, and three-dimensional volume data described later. The medical image data are, for example, three-dimensional image data. The data storage device 3 may be a storage medium (for example, a magnetic storage medium, an electromagnetic storage medium, an optical storage medium, or a semiconductor memory), or a driving device that reads and writes information from or into the storage medium.

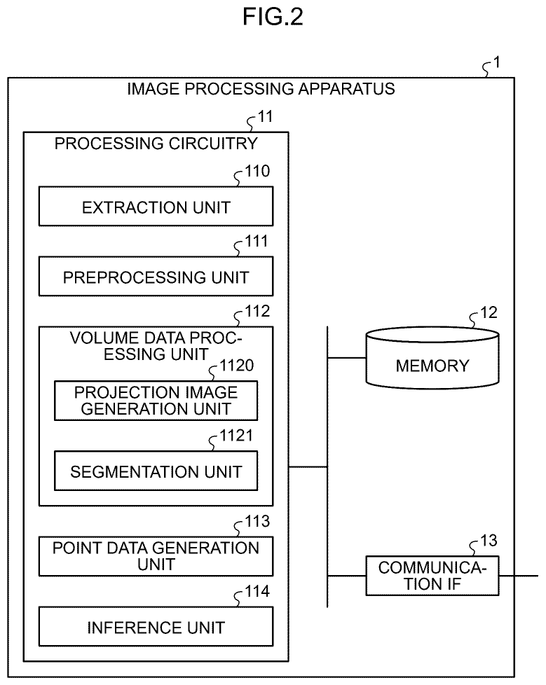

An example of a configuration of the image processing apparatus 1 will be described by reference to FIG. 2. FIG. 2 is a block diagram illustrating the example of the configuration of the image processing apparatus 1 according to the first embodiment.

The image processing apparatus 1 includes processing circuitry 11, a memory 12, and a communication interface (IF) 13. These components are connected so as to be capable of communicating with one another via a bus, which is a common signal transmission path.

The memory 12 is a device to store various data and information. The memory 12 may be a storage medium (for example, a magnetic storage medium, an electromagnetic storage medium, an optical storage medium, or a semiconductor memory) readable by a processor, or may be a driving device that reads and writes data or information from or into the storage medium. The memory 12 stores computer programs that cause the processing circuitry 11 to implement respective functional units.

The communication IF 13 is an interface that communicates various data and information with the respective components included in the image processing system 100. The communication IF 13 communicates various data and information with the operation terminal 2 and the data storage device 3. The communication IF 13 may communicate various data and information with an external device different from the image processing apparatus 1.

The processing circuitry 11 is circuitry that controls the overall operation of the image processing apparatus 1. The processing circuitry 11 includes at least one processor. The processor means a circuit, such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). In a case where the processor is a CPU, the CPU reads and executes each program stored in the memory 12 to implement the respective functions. In a case where the processor is an ASIC, each function is directly incorporated as a logic circuit in the circuit of the ASIC. The processor may be configured as a single circuit or may be configured by combining a plurality of independent circuits with one another. The processing circuitry 11 implements functions of the functional units described later.

In this embodiment, the processing circuitry 11 includes an extraction unit 110, a preprocessing unit 111, a volume data processing unit 112, a point data generation unit 113, and an inference unit 114.

The extraction unit 110 extracts three-dimensional volume data including a region of interest from medical image data. Specifically, the extraction unit 110 extracts a region of interest in, for example, a hepatic blood vessel. The extraction unit 110 removes interference signals in the extracted region of interest in the medical image data by using prior knowledge. The medical image data are represented as three-dimensional volume data, and the extraction unit 110 thus extracts the three-dimensional data including the region of interest. The region of interest has an enhanced signal.

The preprocessing unit 111 preprocesses the three-dimensional volume data extracted by the extraction unit 110. For example, a hepatic blood vessel segmentation task will now be described as an example. The preprocessing unit 111 performs coarse localization of a liver region using a prior-knowledge-based liver segmentation algorithm. The prior-knowledge-based coarse liver segmentation algorithm is not limited, and may be, for example, a conventional algorithm or an artificial intelligence (AI) algorithm. Interference from other anatomical structures in the medical image data is thereby able to be reduced. Subsequently, the preprocessing unit 111 removes a bone region by using a bone segmentation algorithm. Similarly, the bone segmentation algorithm for segmenting the bone region is not limited, and may be, for example, a conventional threshold method or an AI algorithm. Interference from high-intensity signals of bones in the medical image data is thereby able to be eliminated.

The preprocessing unit 111 has been described above to process the liver region, but the embodiment is not limited to this example. For example, the preprocessing unit 111 may segment a region of interest only from the medical image data or may remove interference signals only from the medical image data. For example, the preprocessing unit 111 may segment, from the medical image data, another region (anatomical structure) other than the liver region, or may remove interference signals from a region different from that other region. In a case where the embodiment is applied to an image processing task for medical image data, the image processing task being other than the hepatic blood vessel segmentation task, the preprocessing unit 111 may segment a region of interest by using other prior knowledge, another algorithm, or an AI algorithm, or may remove interference signals therefrom.

The volume data processing unit 112 performs image processing on three-dimensional volume data including a region of interest. Specifically, the volume data processing unit 112 includes a projection image generation unit 1120 and a segmentation unit 1121.

The projection image generation unit 1120 generates, on the basis of the region of interest in the three-dimensional volume data, a projection image that is two-dimensional and buffer data corresponding to the projection image.

For example, the projection image generation unit 1120 generates a maximum intensity projection (MIP) image by projecting the region of interest in the three-dimensional volume data in three directions, onto an axial plane, a sagittal plane, and a coronal plane.

The projection onto the axial plane indicates layer-by-layer projections along the longitudinal axis of the human body and includes structural features of a cross-sectional plane; the projection onto the sagittal plane indicates a lateral view projection along the left-right direction of the human body and includes structural features in the anterior-posterior direction; and the projection onto the coronal plane indicates a frontal view projection along the ventral-dorsal direction of the human body and includes structural features in the superior-inferior direction. The MIP image corresponds to data obtained by visualizing three-dimensional data in the medical image data using a method of visualizing three-dimensional data in medical image data (for example, MIP). By projecting the three-dimensional volume data in the medical image data onto a two-dimensional plane, the MIP image representing a high-density (high signal intensity) anatomical structure is generated.

A method in which the projection image generation unit 1120 generates a two-dimensional projection image on the basis of a region of interest in three-dimensional volume data is not limited to the above described method.

For example, the projection image generation unit 1120 may set different slice intervals for the three-dimensional volume data, segment the three-dimensional volume data at these slice intervals in a specific direction (axial, sagittal, or coronal direction) into a plurality of slice groups, and subsequently execute maximum intensity projection calculation for each slice to obtain an MIP image of the region of interest in the three-dimensional volume data.

The projection image generation unit 1120 may generate an MIP image directly in the region of interest.

The projection image generation unit 1120 thereby generates the MIP image of the region of interest in the three-dimensional volume data.

Furthermore, the projection image generation unit 1120 generates buffer data corresponding to the MIP image of the region of interest. The buffer data include an intensity signal corresponding to the MIP image in the three-dimensional volume data.

The buffer data refer to, for example, a depth buffer (Z buffer). The depth buffer (Z buffer) is buffer data including a plurality of pixels. Each pixel stores one depth value (for example, represented as a floating-point number), and this depth value corresponds to a perpendicular distance from an object surface point in a three-dimensional space to a virtual camera (a Z-axis coordinate along a viewing direction). A two-dimensional projection of the region of interest in the three-dimensional volume data of the medical image data, as well as depth information and spatial relations corresponding to the two-dimensional projection, are thereby able to be obtained.

The buffer data are not limited to the above described example, and may include an intensity signal corresponding to an MIP image in other three-dimensional volume data.

The segmentation unit 1121 segments the region of interest in the MIP image.

Specifically, an example where blood vessels in the liver are segmented will be described hereinafter. For example, the segmentation unit 1121 is capable of performing two-dimensional blood vessel segmentation on an MIP image by using an existing blood vessel segmentation model. Furthermore, the segmentation unit 1121 may perform two-dimensional blood vessel segmentation on an MIP image by using a 2D Med SAM model. For example, the segmentation unit 1121 inputs an MIP image in the above mentioned model and obtains a segmentation result (blood vessels (a region of blood vessels) output from the model. The segmentation unit 1121 thus segments the blood vessels in the MIP image.

On the basis of three-dimensional volume data processed by the volume data processing unit 112, the point data generation unit 113 generates three-dimensional point data representing a region of interest.

Specifically, on the basis of buffer data corresponding to an MIP image generated by the projection image generation unit 1120, the point data generation unit 113 generates three-dimensional point data by mapping a segmentation result (a segmentation result obtained by the segmentation unit 1121) for a region of interest in the MIP image to a three-dimensional space of three-dimensional volume data. The three-dimensional point data are a set of discrete data points represented by coordinates in the three-dimensional space, and each point includes spatial position information and associated attribute values. That is, position information of the three-dimensional point data is typically defined by three coordinate values and the three-dimensional point data are associated with other numerical values, such as gradation intensity, color information, and density values.

The point data generation unit 113 maps a segmentation result for a region of interest generated by the segmentation unit 1121 to a three-dimensional space of three-dimensional volume data by using an MIP image and buffer data corresponding to the MIP image, both generated by the projection image generation unit 1120. Three-dimensional point data of the region of interest representing a three-dimensional discrete scattergram of blood vessels in the liver are thereby generated.

An inference model may be trained. The inference model is, for example, a large-scale segmentation model based on prompt engineering. This large-scale segmentation model understands a user's intent based on a prompt input by the user and completes a segmentation task for a segmentation target.

Specifically, three-dimensional volume data that have been processed by the volume data processing unit 112 and three-dimensional point data that have been generated by the point data generation unit 113 are input to the inference model as prompts in training data, and the inference model is thereby trained. The inference model may be further adjusted using a technique, such as local weight fine-tuning, LoRA, or an adapter-based method. The inference model that has been trained is thereby obtained.

The inference unit 114 performs inference using a trained inference model (an inference model that has been trained (a trained model) and using three-dimensional volume data and three-dimensional point data as prompts. That is, by inputting three-dimensional volume data processed by the preprocessing unit 111 and three-dimensional point data generated by the point data generation unit 113 as prompts into an inference model that has been trained as described above, the inference unit 114 executes a segmentation task for blood vessels in the liver in medical image data and obtains a segmentation result for the blood vessels.

Accordingly, the image processing apparatus 1 according to the first embodiment is applicable to segmentation tasks for complex enhanced signals, enables training time and cost to be reduced, and generalization capability of the model to be improved.

Furthermore, in the image processing apparatus 1 according to the first embodiment, the three-dimensional point data are used as the prompts for the inference model. Therefore, the problem that annotation work for complex enhanced signals requires a long time, high cost, and a large amount of data is able to be avoided. At the same time, automation of a training process and an inference process of the inference model is able to be achieved, manual labor time and cost are able to be reduced, and inference efficiency is able to be improved.

Furthermore, even if the image processing apparatus 1 according to the first embodiment executes a segmentation task for complex enhanced signals of blood vessels or the trachea, high segmentation capability and segmentation accuracy are achieved.

Furthermore, as compared with conventional techniques, the image processing apparatus 1 according to the first embodiment enables improvement of generalization capability of the model and application to any new data region distribution.

### Image Processing Method According to First Embodiment

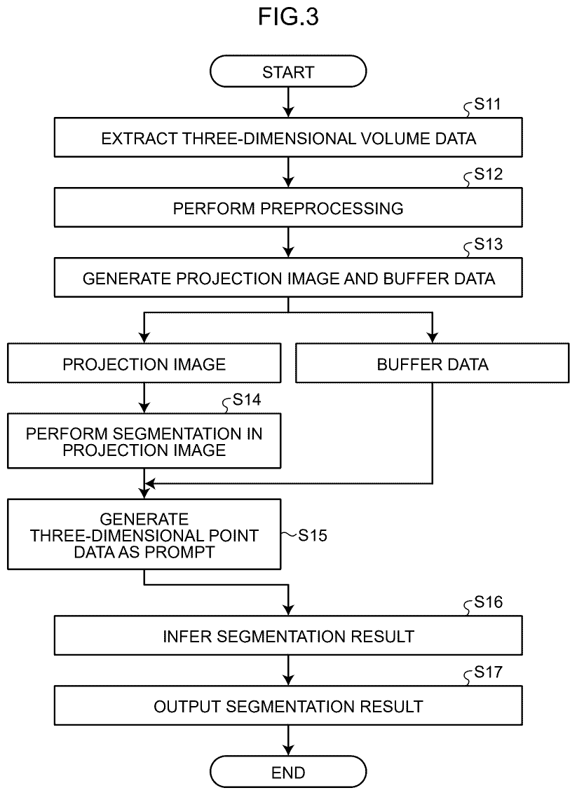

A flow of an image processing method according to the first embodiment will be described hereinafter by reference to FIG. 3. FIG. 3 is a flowchart illustrating the flow of the image processing method according to the first embodiment.

As illustrated in FIG. 3, at Step S11, the extraction unit 110 extracts three-dimensional volume data including a region of interest in a medical image. The flow then proceeds to Step S12.

At Step S12, the preprocessing unit 111 preprocesses the three-dimensional volume data extracted by the extraction unit 110. As the preprocessing, the preprocessing unit 111 may segment the region of interest for the three-dimensional volume data, remove interference signals from the three-dimensional volume data, or perform a combination of the segmentation and the removal. Subsequently, the flow proceeds to Step S13.

At Step S13, the projection image generation unit 1120 of the volume data processing unit 112 generates, on the basis of the region of interest in the three-dimensional volume data processed by the preprocessing unit 111, a projection image (MIP image) that is two-dimensional and buffer data corresponding to the projection image. The projection image generation unit 1120 transmits the generated projection image to the segmentation unit 1121, and transmits the buffer data to the point data generation unit 113.

At Step S14, the segmentation unit 1121 of the volume data processing unit 112 segments the region of interest in the MIP image generated by the projection image generation unit 1120. The segmentation unit 1121 transmits a segmentation result for the region of interest to the point data generation unit 113. Subsequently, the flow proceeds to Step S15.

At Step S15, the point data generation unit 113 receives the buffer data generated by the projection image generation unit 1120 and the segmentation result for the region of interest segmented by the segmentation unit 1121. On the basis of the buffer data corresponding to the MIP image generated by the projection image generation unit 1120, the point data generation unit 113 generates three-dimensional point data by mapping the segmentation result obtained by the segmentation unit 1121 to a three-dimensional space of the three-dimensional volume data. Subsequently, the flow proceeds to Step S16.

At Step S16, by inputting the three-dimensional volume data processed by the preprocessing unit 111 and the three-dimensional point data generated by the point data generation unit 113 as prompts into an inference model (for example, a large-scale segmentation model based on prompt engineering), the inference unit 114 executes a segmentation task for a segmentation target. Subsequently, the flow proceeds to Step S17.

At Step S17, a segmentation result is output. For example, the inference unit 114 causes a display included in the image processing apparatus 1 to display the segmentation result. Furthermore, the inference unit 114 controls the communication IF 13 to output the segmentation result to an external device. The communication IF 13 thereby outputs the segmentation result to the external device. The flow is thereby ended.

### Comparative Example

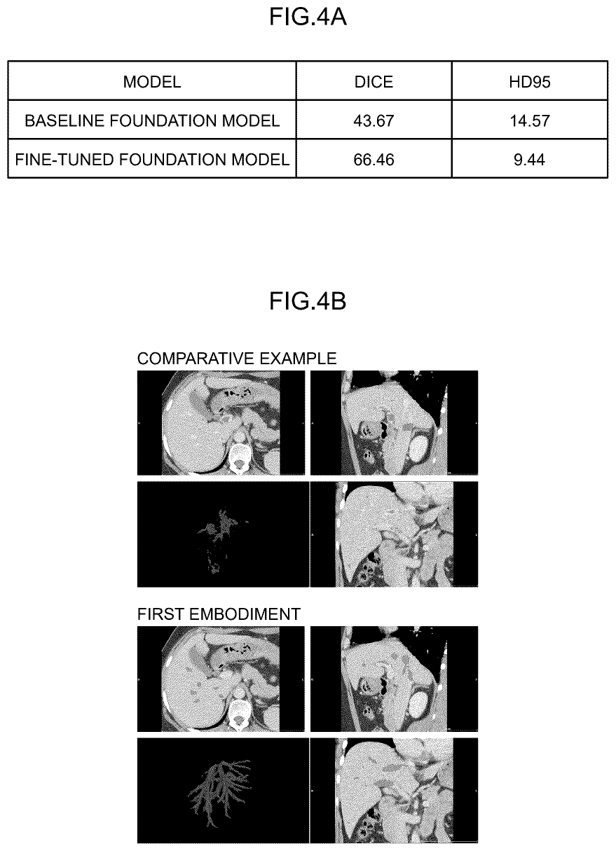

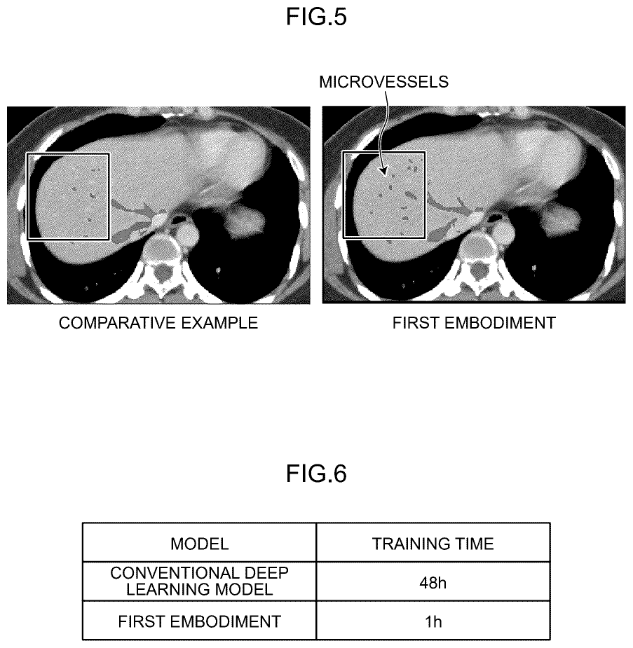

Differences between the first embodiment and a comparative example will be described hereinafter by reference to FIG. 4 to FIG. 6. FIG. 4 is a schematic diagram illustrating the differences between the first embodiment and the comparative example. FIG. 4A is a diagram illustrating differences in evaluation metrics between the first embodiment and the comparative example, FIG. 4B is a diagram illustrating differences in segmentation results between the first embodiment and the comparative example, FIG. 5 is a diagram illustrating differences in segmentation results between the first embodiment and the comparative example, and FIG. 6 is a diagram illustrating differences in evaluation metrics between the first embodiment and the comparative example.

An inference model of the comparative example is, for example, a base model not using the techniques of the first embodiment.

As illustrated in FIG. 4A, in a case where the inference model of the first embodiment is used, both the Dice coefficient and 95% Hausdorff Distance (HD95), which are evaluation metrics, were significantly improved compared with those of the inference model of the comparative example when a segmentation task for hepatic blood vessels was executed on the same medical image data. The Dice coefficient is a degree of agreement between a segmentation result and ground truth (GT), and a higher value indicates higher segmentation accuracy. Furthermore, HD95 is a degree of agreement between a boundary of a segmentation result and a boundary of ground truth (GT), and the smaller the value, the closer the segmentation boundary is to the ground truth boundary.

As described above, accuracy of a segmentation result obtained by the inference model according to the first embodiment is significantly improved as compared with that of the inference model of the comparative example.

As illustrated FIG. 4B, in a case where the inference model according to the first embodiment was used, the segmentation result for blood vessels in a three-dimensional space was significantly improved and continuity, completeness, and accuracy of the segmentation result were improved, as compared with the inference model of the comparative example.

As illustrated in FIG. 5, in a case where the inference model according to the first embodiment is used, when a segmentation task for hepatic blood vessels is executed on the same medical image data, a greater number of microvessels are able to segmented in the medical image data as compared with the ground truth obtained by manual annotation in the comparative example. A segmentation result obtained by the inference model according to the first embodiment is thereby able to be used as ground truth for a subsequent flow, or used as preliminary segmentation input for annotation or ground truth optimization in a subsequent flow.

As illustrated in FIG. 6, in a case where the inference model according to the first embodiment is used, when the inference model is trained using the image processing apparatus and the image processing method according to the first embodiment, the training time is significantly reduced as compared with ground truth obtained by manual annotation in the comparative example.

### Second Embodiment

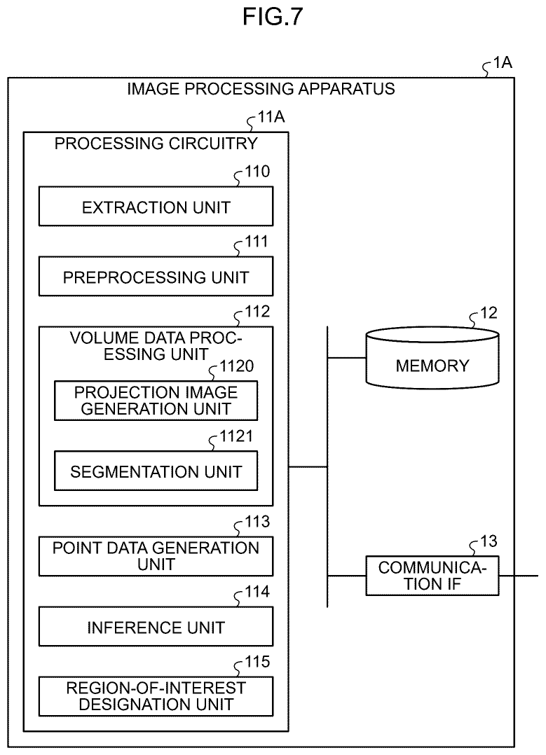

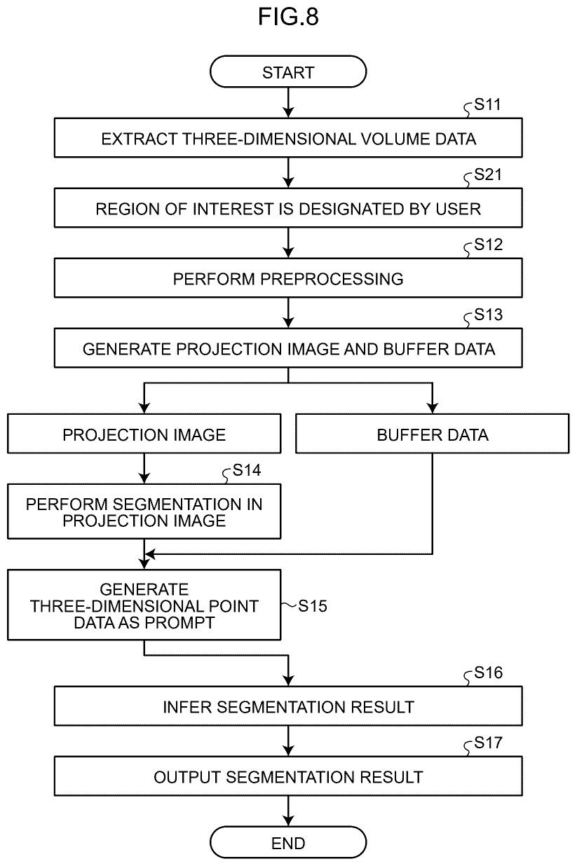

An image processing apparatus 1A according to a second embodiment will be described hereinafter by reference to FIG. 7 and FIG. 8. FIG. 7 is a block diagram illustrating an example of a configuration of the image processing apparatus 1A according to the second embodiment. FIG. 8 is a flowchart illustrating a flow of an image processing method according to the second embodiment. Only differences from the first embodiment will be described with respect to the second embodiment, and description of matters similar to those of the first embodiment will be omitted. Furthermore, the same reference signs will be assigned to components similar to those of the first embodiment and description of the components similar to those of the first embodiment may be omitted.

As illustrated in FIG. 7, the second embodiment is different from the first embodiment in that processing circuitry 11A of the image processing apparatus 1A further includes a region-of-interest designation unit 115.

The region-of-interest designation unit 115 designates a region of interest in three-dimensional volume data on the basis of a user's operation. For example, by operating an operation terminal 2, the user designates a region of interest for three-dimensional volume data in medical image data. A segmentation task is thereby able to be executed even more accurately.

This is because using a conventional inference model generally does not enable satisfactory segmentation of microvessels, resulting in a problem of insufficient segmentation. In contrast, in the image processing apparatus 1A according to the second embodiment, a region including microvessels, which may be undersegmented when a three-dimensional prompt is used, is able to be designated as a region of interest by a user's operation and an inference model is able to be caused to intensively process the designated region of interest. Regions that may be undersegmented, such as microvessels, are thereby able to be segmented satisfactorily.

### Image Processing Method According to Second Embodiment

As illustrated in FIG. 8, the image processing method according to the second embodiment is different from that of the first embodiment in that the image processing method according to the second embodiment further includes Step S21.

At Step S21, on the basis of a user's operation, the region-of-interest designation unit 115 designates a region of interest in three-dimensional volume data extracted by an extraction unit 110. The region-of-interest designation unit 115 transmits the three-dimensional volume data in which the region of interest has been designated, to a preprocessing unit 111.

Other steps of the flow of the image processing method according to the second embodiment are similar to those of the first embodiment and description thereof will thus be omitted.

### Third Embodiment

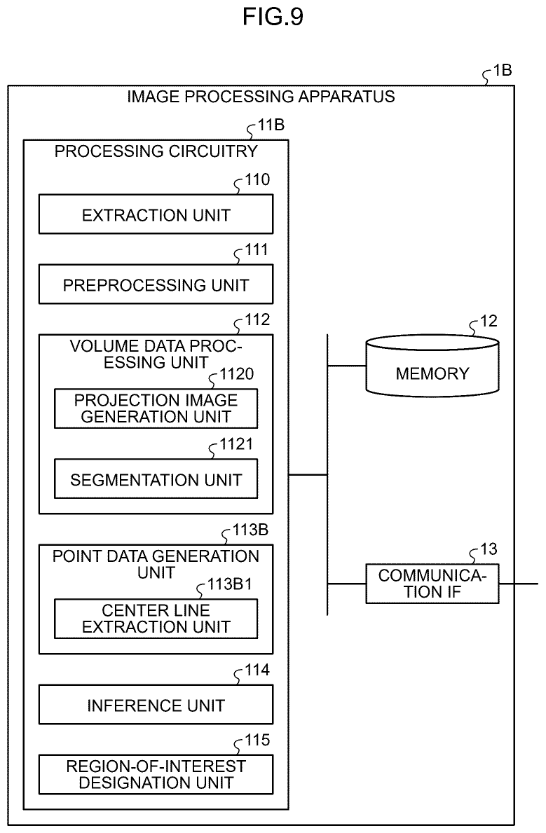

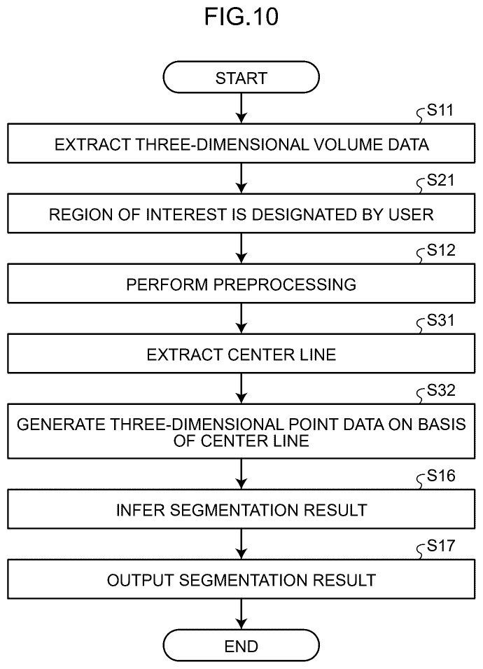

An image processing apparatus 1B according to a third embodiment will be described hereinafter by reference to FIG. 9 and FIG. 10. FIG. 9 is a block diagram illustrating an example of a configuration of the image processing apparatus 1B according to the third embodiment. FIG. 10 is a flowchart illustrating a flow of an image processing method according to the third embodiment. Only differences from the first and second embodiments will be described with respect to the third embodiment, and description of matters similar to those of the first or second embodiment will be omitted. Furthermore, the same reference signs will be assigned to components similar to those of the first or second embodiment and description of the components similar to those of the first or second embodiment may be omitted.

As illustrated in FIG. 9, the third embodiment is different from the first and second embodiments in that the image processing apparatus 1B includes processing circuitry 11B. The third embodiment is different from the first and second embodiments in that a point data generation unit 113B of the processing circuitry 11B further includes a center line extraction unit 113B1.

The center line extraction unit 113B1 extracts a center line from three-dimensional volume data processed by a volume data processing unit 112, and the point data generation unit 113B generates three-dimensional point data on the basis of the center line extracted. Specifically, for example, the center line extraction unit 113B1 generates a core center line in the three-dimensional volume data by using a center line extraction algorithm, and the point data generation unit 113B generates the three-dimensional point data by extracting the center line as a point cloud. As a result, automation of a training process and an inference process of an inference model is able to be improved further, manual labor time and cost are able to be reduced, and inference efficiency is able to be improved.

### Image Processing Method According to Third Embodiment

As illustrated in FIG. 10, the image processing method according to the third embodiment is different from that of the first and second embodiments in that the image processing method according to the third embodiment further includes Steps S31 and S32.

As illustrated in FIG. 10, at Step S11, an extraction unit 110 extracts three-dimensional volume data including a region of interest in a medical image. Subsequently, the flow proceeds to Step S21.

At Step S21, on the basis of a user's operation, a region-of-interest designation unit 115 designates the region of interest in the three-dimensional volume data extracted by the extraction unit 110. The region-of-interest designation unit 115 transmits the three-dimensional volume data in which the region of interest has been designated, to a preprocessing unit 111.

At Step S12, the preprocessing unit 111 preprocesses the region of interest in the three-dimensional volume data designated by the region-of-interest designation unit 115. The preprocessing unit 111 transmits the region of interest, which has been preprocessed, to the point data generation unit 113B. Subsequently, the flow proceeds to Step S31.

At Step S31, the point data generation unit 113B receives the three-dimensional volume data processed by the preprocessing unit 111, and the center line extraction unit 113B1 of the point data generation unit 113B extracts a center line from the three-dimensional volume data processed by the volume data processing unit 112. Subsequently, the flow proceeds to Step S32.

At Step S32, the point data generation unit 113B generates three-dimensional point data on the basis of the center line extracted by the center line extraction unit 113B1. Subsequently, the flow proceeds to Step S16.

At Step S16, by inputting the three-dimensional volume data processed by the preprocessing unit 111 and the three-dimensional point data generated by the point data generation unit 113 as prompts into an inference model (for example, a large-scale segmentation model based on prompt engineering), an inference unit 114 executes a segmentation task for a segmentation target. Subsequently, the flow proceeds to Step S17.

At Step S17, a segmentation result is output. The flow is thereby ended.

### Fourth Embodiment

In the case described above with respect to each of the embodiments, the image processing apparatus executes the various processing described above. However, processing similar to the above described various processing may be executed by an X-ray CT apparatus. Such an embodiment will thus be described as a fourth embodiment.

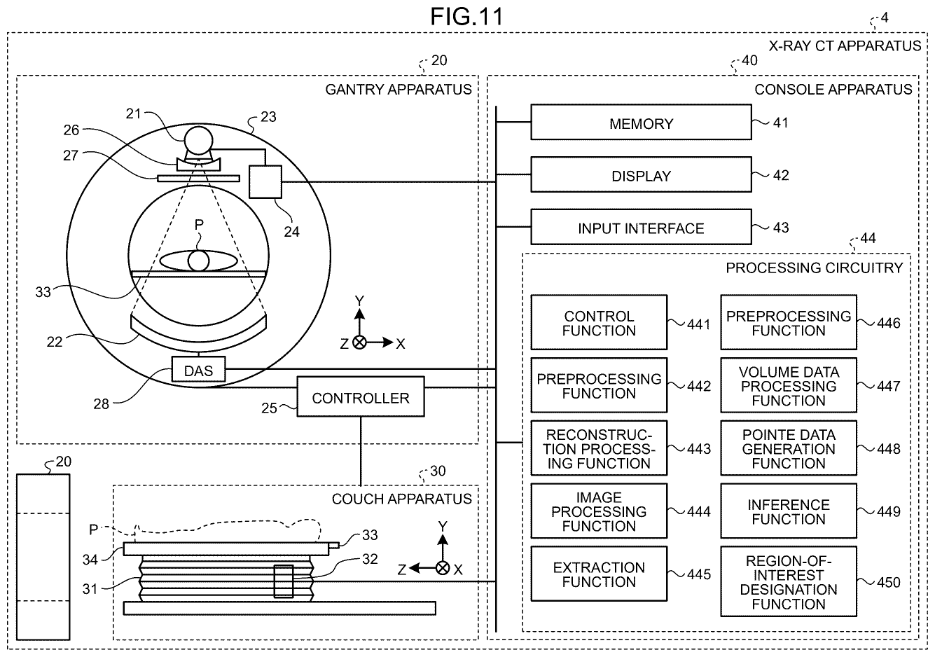

FIG. 11 is a diagram illustrating an example of a configuration of an X-ray CT apparatus according to the fourth embodiment. As illustrated in FIG. 11, an X-ray CT apparatus 4 according to the fourth embodiment has a gantry apparatus 20, a couch apparatus 30, and a console apparatus 40.

In FIG. 11, a rotation axis of a rotating frame 23 in a non-tilted state or a longitudinal direction of a top plate 33 of the couch apparatus 30 will be referred to as a Z-axis direction. The Z-axis direction is an example of a scan axis direction. Furthermore, an axial direction orthogonal to the Z-axis direction and horizontal to a floor surface will be referred to as an X-axis direction. Furthermore, an axial direction orthogonal to the Z-axis direction and the X-axis direction and vertical to the floor surface will be referred to as a Y-axis direction. FIG. 11 depicts the gantry apparatus 20 as viewed from a plurality of directions for description and illustrates a case where the X-ray CT apparatus 4 has one gantry apparatus 20.

The gantry apparatus 20 has an X-ray tube 21, an X-ray detector 22, the rotating frame 23, an X-ray high-voltage generator 24, a controller 25, a wedge 26, a collimator 27, and a data acquisition system (DAS) 28. The gantry apparatus 20 is also referred to as a gantry.

The X-ray tube 21 is a vacuum tube having: a cathode (a filament) that generates thermoelectrons; and an anode (a target) that receives collision of the thermoelectrons and generates X-rays. The thermoelectrons are emitted from the cathode to the anode by application of a high voltage from the X-ray high-voltage generator 24, and the X-ray tube 21 thereby generates X-rays to be emitted to a subject P. For example, the X-ray tube 21 may be a rotating anode X-ray tube that generates X-rays by irradiation of a rotating anode with thermoelectrons.

The X-ray detector 22 detects X-rays that have been emitted from the X-ray tube 21 and have passed through the subject P and outputs a signal corresponding to an amount of the X-rays detected, to the DAS 28. The X-ray detector 22 has, for example, a plurality of detection element rows each having a plurality of detection elements arranged in a channel direction along a single circular arc centered on a focal point of the X-ray tube 21. The X-ray detector 22 has, for example, a structure having the plurality of detection element rows each having the plurality of detection elements arranged in the channel direction, the plurality of detection element rows being arranged in a row direction (slice direction).

For example, the X-ray detector 22 is an indirect-conversion detector having a grid, a scintillator array, and a photosensor array. The scintillator array has a plurality of scintillators. The scintillators each include a scintillator crystal that outputs light having an amount of photons corresponding to an amount of incident X-rays. The grid is disposed on an X-ray incident surface of the scintillator array and has an X-ray shielding plate that absorbs scattered X-rays. The grid may also be called a collimator (one-dimensional collimator or two-dimensional collimator). The photosensor array has a function of performing conversion to an electrical signal corresponding to an amount of light from the scintillators and has, for example, photosensors, such as photodiodes. The X-ray detector 22 may be a direct conversion detector having a semiconductor element that converts incident X-rays into an electrical signal.

The rotating frame 23 is an annular frame that supports the X-ray tube 21 and the X-ray detector 22 opposite to each other and rotates the X-ray tube 21 and the X-ray detector 22 by means of the controller 25. For example, the rotating frame 23 is a casting made of a material including aluminum. The rotating frame 23 may further support, in addition to the X-ray tube 21 and the X-ray detector 22: the X-ray high-voltage generator 24; the wedge 26; the collimator 27; and/or the DAS 28. Furthermore, the rotating frame 23 may further support any of various components not illustrated in FIG. 11. The rotating frame 23 is also referred to as a rotating base or a rotating body. Furthermore, in the gantry apparatus 20, the rotating frame 23 and parts that rotate together with the rotating frame 23 are also referred to as a rotating unit.

The X-ray high-voltage generator 24 has: a high-voltage generator that has an electric circuit including a transformer and a rectifier and generates high voltage to be applied to the X-ray tube 21; and an X-ray controller that controls output voltage according to X-rays generated by the X-ray tube 21. The high-voltage generator may be of a transformer-type or an inverter-type. The X-ray high-voltage generator 24 may be provided on the rotating frame 23 or on a fixed frame not illustrated in the figure.

The controller 25 has: processing circuitry including a central processing unit (CPU); and a driving mechanism, such as a motor and an actuator. The controller 25 controls operation of the gantry apparatus 20 and the couch apparatus 30 by receiving input signals from an input interface 43. For example, the controller 25 controls rotation of the rotating frame 23, tilting of the gantry apparatus 20, and operation of the couch apparatus 30 and the top plate 33. The controller 25 may be provided on the gantry apparatus 20 or provided on the console apparatus 40.

The wedge 26 is a filter for adjusting an amount of X-rays emitted from the X-ray tube 21. Specifically, the wedge 26 is a filter that transmits and attenuates X-rays emitted from the X-ray tube 21 so that a distribution of X-rays emitted from the X-ray tube 21 to the subject P becomes a predetermined distribution. For example, the wedge 26 is a wedge filter or a bow-tie filter, and is a filter made of aluminum that has been processed to achieve a predetermined target angle and/or to have a predetermined thickness.

The collimator 27 includes a plurality of lead plates to narrow an irradiation range of X-rays that have passed through the wedge 26, the plurality of lead plates forming a slit in combination. The collimator 27 may also be called an X-ray diaphragm. Furthermore, FIG. 11 illustrates a case where the wedge 26 is arranged between the X-ray tube 21 and the collimator 27, but the collimator 27 may be arranged between the X-ray tube 21 and the wedge 26 instead. In that case, the wedge 26 transmits and attenuates X-rays emitted from the X-ray tube 21 and having an irradiation range limited by the collimator 27.

The DAS 28 collects signals of X-rays detected by the detection elements included in the X-ray detector 22. For example, the DAS 28 has an amplifier that amplifies electrical signals output from the detection elements, and an A/D converter that converts the electrical signals into digital signals, and the DAS 28 generates detection data.

The data generated by the DAS 28 are transmitted, by optical communication, to a receiver provided in a non-rotating portion (for example, a fixed frame not illustrated in FIG. 11) of the gantry apparatus 20 from a transmitter having a light emitting diode (LED) provided on the rotating frame 23, the receiver having a photodiode, and are transferred to the console apparatus 40. The non-rotating portion is, for example, a fixed frame that rotatably supports the rotating frame 23. The data from the rotating frame 23 to the non-rotating portion of the gantry apparatus 20 are not necessarily transmitted by optical communication, and any non-contact type data transmission method may be adopted or a contact-type data transmission method may be adopted.

The couch apparatus 30 is an apparatus on which the subject P to be imaged is placed and which moves the subject P, and has a base 31, a couch driving device 32, the top plate 33, and a support frame 34. The base 31 is a housing that supports the support frame 34 movably in a vertical direction. The couch driving device 32 is a driving mechanism that moves the top plate 33 having the subject P placed thereon, in a longitudinal direction of the top plate 33, and includes a motor and an actuator. The top plate 33 provided on an upper surface of the support frame 34 is a plate where the subject P is placed. The couch driving device 32 may move, in addition to the top plate 33, the support frame 34 along the longitudinal direction of the top plate 33.

The console apparatus 40 has a memory 41, a display 42, the input interface 43, and processing circuitry 44. The console apparatus 40 is described to be separate from the gantry apparatus 20, but the console apparatus 40 or some of components of the console apparatus 40 may be included in the gantry apparatus 20.

The memory 41 is implemented by, for example: a semiconductor memory element, such as a random access memory (RAM) or a flash memory; a hard disk; or an optical disk. The memory 41 stores, for example, projection data and CT image data. Furthermore, for example, the memory 41 stores programs for the circuitry included in the X-ray CT apparatus 4 to implement various functions. The memory 41 may be implemented by a server group (a cloud) connected to the X-ray CT apparatus 4 via a network.

The display 42 displays various information. For example, the display 42 displays various images generated by the processing circuitry 44 and displays a graphical user interface (GUI) for receiving various operation from an operator. For example, the display 42 is a liquid crystal display or a cathode ray tube (CRT) display. The display 42 may be of a desktop type, or may be formed of a tablet terminal capable of wirelessly communicating with the main body of the console apparatus 40. Furthermore, the display 42 is an example of a display unit.

The input interface 43 receives various input operation from an operator, converts the input operation received, into electrical signals, and outputs the electrical signals to the processing circuitry 44. Furthermore, for example, the input interface 43 receives, from an operator, input operation for scan conditions, reconstruction conditions for reconstruction of CT image data, and image processing conditions for generation of post-processed images from the CT image data.

For example, the input interface 43 is implemented by any of: a mouse and a keyboard; a trackball; switches; buttons; a joystick; a touchpad enabling input operation by a touch on an operation surface; a touchscreen having a display screen and a touchpad that have been integrated; a non-contact input circuit using an optical sensor; and a voice input circuit. The input interface 43 may be provided on the gantry apparatus 20. Furthermore, the input interface 43 may be formed of a tablet terminal capable of wirelessly communicating with the main body of the console apparatus 40. Furthermore, the input interface 43 does not necessarily include physical operation parts, such as a mouse and a keyboard. Examples of the input interface 43 include an electrical signal processing circuit that receives an electrical signal corresponding to input operation from an external input device provided separately from the console apparatus 40 and outputs the electrical signal to the processing circuitry 44.

The processing circuitry 44 controls the overall operation of the X-ray CT apparatus 4. For example, the processing circuitry 44 executes a control function 441, a preprocessing function 442, a reconstruction processing function 443, an image processing function 444, an extraction function 445, a preprocessing function 446, a volume data processing function 447, a point data generation function 448, an inference function 449, and a region-of-interest designation function 450. For example, respective processing functions executed by the control function 441, the preprocessing function 442, the reconstruction processing function 443, the image processing function 444, the extraction function 445, the preprocessing function 446, the volume data processing function 447, the point data generation function 448, the inference function 449, and the region-of-interest designation function 450, which are components of the processing circuitry 44 illustrated in FIG. 11, are recorded, in the form of programs executable by a computer, in the memory 41. The processing circuitry 44 is, for example, a processor, reads these programs from the memory 41, and implements the functions corresponding to the read programs by executing the read programs. In other words, the processing circuitry 44 that has read the programs has the functions illustrated inside the processing circuitry 44 in FIG. 11.

FIG. 11 illustrates the case where the respective processing functions of the control function 441, the preprocessing function 442, the reconstruction processing function 443, the image processing function 444, the extraction function 445, the preprocessing function 446, the volume data processing function 447, the point data generation function 448, the inference function 449, and the region-of-interest designation function 450 are implemented by the processing circuitry 44, but the embodiment is not limited to this case. For example, the processing circuitry 44 may be formed of a combination of a plurality independent processors, and the processing functions may be implemented by these processors executing the respective programs. Furthermore, any of the processing functions of the processing circuitry 44 may be implemented by being distributed to a plurality of processing circuits or integrated into a single processing circuit, as appropriate.

The control function 441 controls various processes on the basis of input operation received from an operator via the input interface 43. Specifically, the control function 441 controls CT scanning performed at the gantry apparatus 20. For example, the control function 441 controls collection processing for results of counting at the gantry apparatus 20, by controlling operation of the X-ray high-voltage generator 24, the X-ray detector 22, the controller 25, the DAS 28, and the couch driving device 32. For example, the control function 441 controls collection processing for projection data in each of positioning scanning for collecting positioning images (scannogram images) and imaging (main scanning) for collecting images to be used for diagnosis.

Furthermore, the control function 441 causes the display 42 to display images based on various image data stored in the memory 41.

The preprocessing function 442 generates projection data by performing preprocessing, such as logarithmic transformation, offset correction, sensitivity correction between channels, beam hardening correction, scatter correction, and/or dark count correction, with respect to detection data output from the DAS 28. Data resulting from the preprocessing of the detection data are also referred to as raw data. Furthermore, the detection data before the preprocessing or the raw data after the preprocessing are also referred to as projection data.

The reconstruction processing function 443 generates CT image data by performing reconstruction processing using a filtered back projection method or a successive approximation reconstruction method, on the projection data generated by the preprocessing function 442. The reconstruction processing function 443 stores the reconstructed CT image data into the memory 41. The CT image data stored in the memory 41 are, for example, three-dimensional medical image data depicting an anatomical structure, such as the liver of the subject P.

On the basis of input operation received from an operator via the input interface 43, the image processing function 444 converts the CT image data generated by the reconstruction processing function 443 into image data, such as a tomographic image of any cross section or a three-dimensional image resulting from rendering processing, by using a publicly known method. The image processing function 444 stores the converted image data into the memory 41.

The extraction function 445, the preprocessing function 446, the volume data processing function 447, the point data generation function 448, the inference function 449, and the region-of-interest designation function 450 perform processing similar to the processing executed by the image processing apparatus in any one of the above described first to third embodiments by using the three-dimensional CT image data stored in the memory 41.

For example, the extraction function 445 executes processing similar to the processing executed by the extraction unit 110 described above. The preprocessing function 446 executes processing similar to the processing executed by the preprocessing unit 111 described above. The volume data processing function 447 executes processing similar to the processing executed by the volume data processing unit 112 described above. The point data generation function 448 executes processing similar to the processing executed by the above described point data generation unit 113 or point data generation unit 113B. The inference function 449 executes processing similar to the processing executed by the inference unit 114 described above. The region-of-interest designation function 450 executes processing similar to the processing executed by the region-of-interest designation unit 115 described above.

Therefore, the X-ray CT apparatus 4 according to the fourth embodiment achieves effects similar to the effects achieved by the above described embodiments.

In the description of each embodiment, the enhanced signals have been described as being signals having higher structural intensities than surrounding signals, but without being limited to such signals, the enhanced signals may instead be signals having lower structural intensities than surrounding signals.

Furthermore, in the description of each embodiment, the projection image generated by the projection image generation unit 1120 has been described as being a maximum intensity projection, but without being limited to the maximum intensity projection, the projection image generated by the projection image generation unit 1120 may be a minimum intensity projection.

Furthermore, on the basis of three-dimensional volume data processed by a volume data processing unit, a point data generation unit may arrange three-dimensional point data within a region of interest in the three-dimensional volume data.

Furthermore, in the description of each embodiment, the case where the region of interest includes a blood vessel has been described as an example. In such a case, a point data generation unit arranges three-dimensional point data along blood vessels. However, without being limited to this case, a region of interest may be another organ, tissue, or structure, such as a bone. In such a case, a point data generation unit arranges three-dimensional point data along such a region of interest.

Furthermore, in a case where a region of interest includes a blood vessel, a point data generation unit may treat a branch point of the blood vessel as a node and arrange three-dimensional point data at an end of the blood vessel.

Furthermore, in the above-described embodiments, the processing circuitry is not limited to being implemented by a single processor. The processing circuitry may be configured by combining a plurality of independent processors, and these processors may implement the respective processing functions by executing programs. Furthermore, the respective processing functions of the processing circuitry may be implemented by being distributed to a plurality of processing circuits or integrated into a single processing circuit. Furthermore, the respective processing functions of the processing circuitry may also be implemented by a combination of hardware, such as a circuit, and software. Furthermore, the case where the programs corresponding to the respective processing functions are stored in a single storage circuit has been described above as an example, but the embodiments are not limited to this example. For example, a configuration, in which the programs corresponding to the respective processing functions are distributed and stored in a plurality of storage circuits, and the processing circuitry reads and executes the programs from the respective storage circuits, may be adopted.

Furthermore, with respect to the above-described embodiments, the case where each functional unit in the present specification is implemented by the processing circuitry has been described as an example, but the embodiments are not limited to this example. For example, each functional unit in the present specification may be implemented solely by hardware, solely by software, or by a combination of hardware and software, instead of being implemented by the processing circuitry described with respect to the embodiments.

The term, “processor”, used above in the description of the embodiments means, for example, a circuit, such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). Instead of being stored in a storage circuit, a computer program may be directly incorporated in the circuit of a processor. In this case, by reading and executing the program incorporated in the circuit, the processor implements a function. Furthermore, each processor in the embodiments is not limited to being configured as a single circuit, and a plurality of independent circuits may be combined to be configured as one processor and implement their functions.

Furthermore, a computer program product includes a computer program executed by a processor, and this computer program is provided by being incorporated in a read only memory (ROM) or a storage circuit beforehand. This computer program may also be provided as a file in an installable format or executable format for those devices by being recorded in a computer-readable non-transitory storage medium, such as a compact disk ROM (CD-ROM), a floppy disk (FD), a CD recordable (CD-R), or a digital versatile disk (DVD). The computer program may also be provided or distributed by being stored on a computer connected to a network, such as the Internet, and being downloaded via the network. For example, the computer program is configured as a module or modules including the above described processing functions. As to actual hardware, by a CPU reading and executing the computer program from a storage medium, such as a ROM, each module is loaded and generated on a main storage device.

With respect to the above described embodiments, the components of each apparatus have been functionally and conceptually illustrated in the drawings and are not necessarily configured physically as illustrated in the drawings. That is, specific forms of distribution and integration of each apparatus are not limited to those illustrated in the drawings, and all or part of each apparatus may be configured to be distributed or integrated functionally or physically in any units, according to various loads and use situations. Furthermore, all or any part of the processing functions executed in each apparatus may be implemented by a CPU and a program analyzed and executed by the CPU, or implemented as hardware by wired logic.

Furthermore, of the processing described above with respect to the embodiments, all or part of the processing described as being performed automatically may be performed manually, or all or part of the processing described as being performed manually may be performed automatically by a publicly known method. In addition, the processing procedures, control procedures, specific names, and information including various data and parameters, which have been described above and illustrated in the drawings, may be modified in any way unless otherwise specified.

Furthermore, various data handled in the present specification are typically digital data.

Although some embodiments of the present invention have been described, these embodiments are presented as examples and are not intended to limit the scope of the invention. These embodiments may be implemented in various other forms, and various omissions, substitutions, and modifications may be made without departing from the spirit of the invention. These embodiments and modifications thereof are included in the scope and spirit of the invention, as well as within the scope of the invention as set forth in the claims and their equivalents.

At least one of the embodiment described above enables inference efficiency to be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing method, including:
a point data generation step of generating, on the basis of three-dimensional volume data including a region of interest, three-dimensional point data representing the region of interest; and
an inference step of inferring the region of interest in the three-dimensional volume data, by inputting the three-dimensional volume data and the three-dimensional point data as prompts into an inference model.

2. The image processing method according to claim 1, further comprising:
a volume data processing step including: a projection image generation step of generating, on the basis of the region of interest in the three-dimensional volume data, a projection image that is two-dimensional and buffer data corresponding to the projection image; and a segmentation step of segmenting the region of interest in the projection image, wherein
in the point data generation step, the three-dimensional point data are generated on the basis of the three-dimensional volume data that have been processed in the volume data processing step.

3. The image processing method according to claim 2, wherein the buffer data include an intensity signal corresponding to the projection image in the three-dimensional volume data.

4. The image processing method according to claim 2, wherein in the point data generation step, on the basis of the buffer data, a segmentation result for the region of interest in the projection image is mapped to a three-dimensional space of the three-dimensional volume data to generate the three-dimensional point data.

5. The image processing method according to claim 2, further comprising:
a preprocessing step of preprocessing the three-dimensional volume data, wherein
in the inference step, the three-dimensional volume data that have been preprocessed and the three-dimensional point data are used as the prompts.

6. The image processing method according to claim 5, wherein in the preprocessing step, the region of interest is segmented from the three-dimensional volume data, and an interference signal is removed therefrom.

7. The image processing method according to claim 2, wherein in the projection image generation step, the projection image is generated by using at least one of: a method of generating the projection image in each of a plurality of directions; a method of generating the projection image by setting a slice interval; and a method of generating the projection image in the region of interest.

8. The image processing method according to claim 1, further including a region-of-interest designation step of designating the region of interest in the three-dimensional volume data on the basis of a user's operation.

9. The image processing method according to claim 1, wherein the inference model is a large-scale segmentation model based on prompt engineering.

10. The image processing method according to claim 1, wherein an enhanced signal of the region of interest is a signal having a structural intensity lower or higher than that of surrounding signals.

11. The image processing method according to claim 1, wherein the projection image is a projection image obtained by a maximum intensity projection or a minimum intensity projection.

12. The image processing method according to claim 1, wherein in the point data generation step, a center line is extracted from the three-dimensional volume data, and the three-dimensional point data are generated on the basis of the center line.

13. The image processing method according to claim 1, wherein in the point data generation step, the three-dimensional point data are arranged in the region of interest.

14. The image processing method according to claim 1, wherein
the region of interest includes a blood vessel, and
in the point data generation step, the three-dimensional point data are arranged along the blood vessel.

15. The image processing method according to claim 1, wherein
the region of interest includes a blood vessel, and
in the point data generation step, a branch point of the blood vessel is treated as a node, and the three-dimensional point data are arranged at an end of the blood vessel.

16. The image processing method according to claim 2, wherein
the buffer data include information of the three-dimensional volume data, the information being along a first direction orthogonal to the projection image, and
in the point data generation step, the three-dimensional point data are generated by mapping the segmentation result to the three-dimensional space by using the information of the three-dimensional volume data included in the buffer data, the information being along the first direction.

17. An image processing apparatus, comprising:
processing circuitry that generates, on the basis of three-dimensional volume data including a region of interest, three-dimensional point data associated with the region of interest, and infers the region of interest in the three-dimensional volume data, by inputting the three-dimensional volume data and the three-dimensional point data as prompts into an inference model.

18. An X-ray CT apparatus, comprising:
processing circuitry that generates, on the basis of three-dimensional volume data including a region of interest, three-dimensional point data associated with the region of interest, and infers the region of interest in the three-dimensional volume data, by inputting the three-dimensional volume data and the three-dimensional point data as prompts into an inference model.
